(19) United States
(12) Patent Application Publication
(10) Pub. No.: US 2022/0396756 A1
(43) Pub. Date: Dec. 15, 2022

(54) BIOREACTOR CHAMBER

(71) Applicant: Oxford University Innovation Limited, Oxford (GB)

(72) Inventor: Pierre-Alexis MOUTHUY, Oxford (GB)

(21) Appl. No.: 17/762,823

(22) PCT Filed: Sep. 23, 2020
(86) PCT No.: PCT/GB2020/052301

§ 371 Date: Mar. 23, 2022

(30) Foreign Application Priority Data
Sep. 24, 2019 (GB) 1913745.4

(51) Int. Cl. C12M 1/42; C12M 1/12; C12M 1/00

(52) U.S. Cl. CPC C12M 35/04; C12M 25/02; C12M 25/14; C12M 23/26; C12M 23/22

(57) ABSTRACT

A bioreactor chamber (1) including a first end block (4), a second end block (6) and a flexible membrane (2). The flexible membrane (2) extends between the first end block (4) and the second end block (6) and defines a cavity (10) bounded by at least the flexible membrane (2). The cavity (10) is arranged to receive a substrate, for growing a culture on the substrate or a biomaterial for testing the biomaterial.

This invention relates to a bioreactor chamber for cell culture, tissue engineering and biomaterial testing.

When carrying out cell or tissue engineering within a bioreactor chamber, it is important that conditions within the chamber are carefully controlled. Mechanical stimulation may be applied to tissue structures during growth, for example by using robotic actuator systems, in order to provide mechanical cues to the tissue structure, which is thought to affect the growth of the tissue. For this purpose, bioreactor chambers may include actuators which are used to apply unilateral forces directly to the tissue structures.

However, conventional bioreactor chambers are rigid and often contain only linear actuators, resulting in only a limited range of mechanical stimulation being able to be applied. This limited range of motion does not allow the successful production of functional grafts. Furthermore the experiments that can be carried out on an engineered tissue within a conventional bioreactor chamber are very limited, and the bioreactor chamber must be opened in order to carry out tests on a tissue engineered structure, owing to the linear actuators forming part of the bioreactor chamber, thus bringing an end to a particular experiment.

There is a need to provide an improved bioreactor chamber.

From a first aspect, the invention provides a bioreactor chamber comprising:
a first end block;
a second end block; and
a flexible membrane, extending between the first end block and the second end block, to define a cavity bounded by at least the flexible membrane;
wherein the cavity is arranged to receive:
   a substrate, for growing a culture on the substrate; or
   a biomaterial for testing the biomaterial.

The invention provides a bioreactor chamber in which a cavity is formed by a membrane between first and second end blocks. The cavity (and thus the bioreactor chamber) is suitable for growing a culture, e.g. a cell culture or a tissue culture (e.g. a tissue structure) on the substrate within the cavity. Additionally or alternatively the bioreactor chamber may also be suitable for testing biomaterials in the cavity.

Thus it will be seen that, in accordance with the invention, a bioreactor chamber is provided that may be actuated to perform many different kinds of motion so as to apply a variety of mechanical stimulations (e.g. tension, compression, torsion and/or shear stresses) to a substrate (and thus to the culture being grown) or to a biomaterial which is being tested. The bioreactor chamber of the present invention may thus be able to be used with (and thus combine) both uniaxial and multiaxial actuation systems. This is possible owing to the cavity of the bioreactor chamber being bounded and defined, at least in part, by a membrane which is flexible.

The present invention, by providing a cavity that is defined by a membrane, also helps to provide a bioreactor chamber that is a self-contained unit, which may be actuated from outside of the bioreactor chamber. The first end block and/or the second end block may be connected to parts of an actuator system, in order to actuate the substrate contained within the cavity, or the biomaterial. The bioreactor chamber may thus be separable from an actuator system without interfering with the cavity or the substrate, or biomaterial, within the cavity. This may allow a larger range of experiments and tests to be carried out on a culture (e.g. a cell or tissue culture), or a biomaterial, within the bioreactor chamber, without opening the bioreactor chamber, and therefore ending a particular experiment. For example, this may allow the bioreactor chamber to be removed from an actuator system and transported to a microscope for inspection of the culture or biomaterial.

In some embodiments, the cavity is arranged to receive a substrate for growing a culture. In these embodiments, the bioreactor chamber is suitable for growing a culture. Thus, in a set of preferred embodiments, the bioreactor chamber further comprises a substrate, arranged within the cavity for growing a culture on the substrate. The substrate may comprise any suitable substrate for growing a culture. In a set of embodiments the substrate comprises a scaffold.

The culture may comprise any suitable and desired culture, e.g. a cell culture or a tissue culture (e.g. a tissue structure). In one set of embodiments the culture comprises a fibroblast, e.g. a tendon or ligament fibroblast. In one set of embodiments the culture comprises one or more of an osteoblast, a chondrocyte, stem cells and immune cells.

In another set of embodiments, the cavity is arranged to receive a biomaterial for testing the biomaterial. Thus preferably the bioreactor chamber comprises a biomaterial, arranged within the cavity, for testing the biomaterial in the cavity. The biomaterial may comprise any suitable and desired biomaterial, e.g., an (e.g. organic) biological material, e.g. a culture grown on a substrate as described herein. The biomaterial may comprise a tissue structure, for example as grown according to the methods disclosed herein.

Thus the biomaterial may comprise a substrate, optionally the biomaterial comprises a scaffold as described herein. The biomaterial may comprise a natural or a synthetic biomaterial, e.g. one or more of an electrospun material (e.g. a filament formed from electrospun fibres), a 3D printed biomaterial, a hydrogel, a sponge, etc. The biomaterial may comprise a (e.g. multiphasic) scaffold, e.g. that comprises a gradient of composition and/or properties (such as of hydroxyapatite content, e.g. to mimic the transition between soft and hard tissues).

The cavity may have any suitable three-dimensional shape. The flexible membrane is, at least in part, arranged to form the three-dimensional shape of the cavity. Thus, although the flexible membrane is a thin sheet that may not itself (the sheet) contain a cavity, it can be shaped to construct a three-dimensional volume with an outer surface defined by the membrane. For example, two edges of the flexible membrane may be joined together, e.g. by heat welding, to create a three-dimensional volume defined by the membrane (e.g. having a tubular shape).

The flexible membrane, e.g. on its own, may form the entire cavity, but the membrane may still extend between (e.g. be connected to) the end plates. For example, the flexible membrane may be pinched by each of the first and second end blocks (or the ends of the membrane may be heat sealed together before being connected to the first and second end blocks), forming a cavity which is bounded entirely by the flexible membrane.

However, in a preferred set of embodiments, the cavity is bounded by the first end block, the second end block and the flexible membrane. Thus, in at least preferred embodiments, the membrane meets the first end block and the second end block such that an inner three-dimensional volume (i.e. the cavity) is surrounded across its entire surface, by either the membrane, the first end block or the second end block. The flexible membrane thus, in at least preferred embodiments, forms an outer (e.g. side) wall of the cavity, with each of the first end block and the second end block forming another outer (e.g. end) wall of the cavity respectively.

In some embodiments, the flexible membrane is arranged into a substantially tubular shape, thus creating a cavity that has a substantially tubular shape (e.g. with the membrane forming the side wall(s) of the cavity). Preferably, the (e.g. substantially tubular) cavity has end walls defined by the first end block and the second end block. This tubular shape provides a simple bioreactor chamber design with a cavity of a shape that is particularly well suited to cell culture, e.g. tissue-engineering.

In some embodiments, it may be desirable to grow a culture, e.g. a cell or tissue culture around a central passage, or secondary cavity, within the cavity of the bioreactor chamber. This may help, for example, to form a cell or tissue culture (e.g. structure) having a lumen extending therethrough. Therefore, in some embodiments, the bioreactor chamber additionally comprises a central passage, within the cavity, extending between the first end block and the second end block. For example, the flexible membrane may be a double-walled membrane, defining the cavity between the walls of the flexible membrane, and further defining the central passage within the inner wall of the flexible membrane. The central passage is preferably isolated from the cavity.

The flexible membrane may be any suitable material. A suitable material may be a material able to undergo a variety of mechanical actuations without yielding or failing (i.e. breaking). This may, for example, require that the material of the flexible membrane has a Young's modulus sufficiently low to allow the membrane to undergo a variety of motions without yielding. Preferably the flexible material provides (e.g. very) low resistance to loading. This helps to improve the actuation of the substrate or biomaterial, since the flexible material will be effectively non-load bearing, which thus helps to transfer any external mechanical actuations or stresses to the substrate (and thus to a culture which is grown on the substrate) or to a biomaterial. Preferably the flexible membrane is impermeable.

Preferably, the flexible membrane is formed of polyurethane, e.g. a polyurethane sheet that is formed as (e.g. rolled into) a tubular shape. For example, two edges of the membrane may be sealed to each other to form a tubular shape, e.g. such that the membrane is sealed along the axial length of the resulting tubular shape.

In some embodiments the flexible membrane is at least partly transparent. Thus the flexible membrane may be sufficiently permeable to light such that a culture (e.g. structure) or biomaterial within the cavity, the cavity defined, at least in part, by the flexible membrane, is visible from the exterior of the cavity. In some embodiments, the flexible membrane may have a thickness less than 100 microns, e.g. less than 50 microns.

The flexible membrane extends between, and thus is preferably connected to, the first end block and/or the second end block, to form the cavity. For example, the flexible membrane may be fixed in permanent connection to the first end block and/or the second end block. In preferred embodiments, the flexible membrane is clamped to or in the first end block and/or the second end block. In some embodiments the flexible membrane is sealingly connected to the first end block and/or the second end block. Thus the flexible membrane may be connected to each of the first end block and/or the second end block in a manner that forms a sealed connection between the membrane and each block, such that at this connection point the cavity is sealed.

There are many types of sealing connection which may be used in accordance with embodiments of the present invention. In one preferred embodiment, the first end block comprises an outer member and an inner member, wherein the flexible member is clamped between the outer member and the inner member. It will be understood by the skilled person that the feature described above in reference to the first end block may be (and preferably is) present equally in the second end block. In some embodiments, the first end block and the second end block are substantially identical.

In some embodiments, the first end block further comprises a ring, and the flexible membrane passes through an aperture in the inner member, through the ring, to be folded back on itself to surround the ring, and, e.g., passes back through the aperture in the inner member. Thus, for example, the inner member of the first (and/or second) end block comprises an aperture. The flexible membrane, the outer member and the inner member are attached together, and the ring is clamped between the outer member and the inner member. Thus, in this embodiment, the flexible member is clamped between the outer member and the inner member using the ring. The ring helps to create a seal between the flexible membrane and the first (and/or second) end block, thus helping to seal the cavity. In some preferred embodiments, the ring is a rigid ring.

The inner member may comprise a recessed portion, wherein the recessed portion comprises the aperture. The recessed portion may be sized to at least partially contain the ring. When the inner member and the outer member are attached together, with the ring clamped between them, the recessed portion may receive the ring, thus accommodating the ring and allowing the inner member and the outer member to be attached more closely together. This may help to provide an improved seal.

The first end block may comprise a deformable seal (e.g. an O-ring), positioned between the ring and the inner member. This may provide an improved seal. Additionally, or alternatively, the first end block may comprise a deformable seal (e.g. an O-ring), positioned between the outer member and the ring. Since the flexible membrane surrounds the ring, the deformable seal may be positioned at the point at which the flexible membrane passes around the exterior of the ring to contact the flexible membrane. The deformable seal(s) may be rubber, silicone or Teflon®. By contacting the flexible membrane, at the point at which the flexible membrane passes around the exterior of the ring, the deformable seal helps to provide an improved seal and reduces wear to the flexible membrane.

Although it is described above that the connection of the flexible membrane to the end blocks may be a sealed connection, this does not necessarily entail that the cavity is sealed across its entire outer surface. In some embodiments, the cavity comprises a fluid inlet, e.g. for cell culture medium to be introduced into the cavity. The inlet may be suitable for cell culture medium perfusion. The first end block and/or the second end block may comprise the fluid inlet (e.g. in some examples each end block may comprise an inlet), e.g. for supplying cell culture medium to the cavity.

In some preferred embodiments, the first end block comprises an inlet and the second end block comprises an outlet, each fluidly connected to the cavity. Preferably, the inlet and the outlet are arranged to be positioned offset from each other on opposing sides of the cavity, i.e. such that they are not directly opposite each other. This helps to cause any fluid which is input to the cavity to have to pass across the substrate or biomaterial to the outlet.

In some embodiments, the substrate or biomaterial may sit (e.g. freely) within the cavity. In some embodiments, the substrate comprises a scaffold, e.g. a structured material, for providing an underlying support to the culture. Preferably the substrate (e.g. scaffold) or biomaterial extends between the first end block and the second end block. Preferably, the biomaterial or substrate (e.g. scaffold) is connected to the first end block and the second end block. This helps to allow direct manipulation of the substrate (e.g. scaffold) (and hence of the culture (e.g. tissue structure) growing on the substrate (e.g. scaffold)) or of the biomaterial, by manipulation of the end blocks to which the substrate (e.g. scaffold) or the biomaterial is connected. In some embodiments, the substrate (e.g. scaffold) or biomaterial is fixed to the first end block and/or the second end block. The substrate (e.g. scaffold) or biomaterial may be fixed to the first end block and/or the second end block using resin. This may result in a need to cut the biomaterial or substrate (e.g. scaffold) in order to remove the culture or biomaterial, thus making the bioreactor chamber single use.

The substrate may comprise any suitable and desired material, such as a fluid comprised of beads, or a substantially flat surface. In embodiments in which the substrate comprises a scaffold, the scaffold may comprise any suitable material and may be produced using any suitable construction method. The scaffold may, for example, be produced by electrospinning, solvent casting, freeze-drying, 3D printing, or produced from hydrogels. The scaffold may alternatively be a tissue explant (e.g. a living tissue sample removed from a living human or animal).

The first end block and/or the second end block may be adapted to fit any specific type of substrate (e.g. scaffold). In some embodiments, the scaffold comprises an electrospun fibre, e.g. formed from a plurality of (e.g. electrospun) filaments. Optionally, the filaments may be substantially aligned. In some embodiments, the scaffold is porous. The electrospun fibres may, for example, be formed from an electrospinning solution, made by dissolving PCL (polycaprolactone) into HFIP (1,1,1,3,3,3-hexafluoroisopropanol).

The inlet described above allows cells and/or cell culture media required for growth of a culture to be supplied to the substrate in the cavity. Thus, from a second aspect, the invention provides a method of growing a culture using a bioreactor chamber, wherein the bioreactor chamber comprises:
a first end block;
a second end block;
a flexible membrane, extending between the first end block and the second end block, to define a cavity bounded by at least the flexible membrane; and
a substrate, arranged within the cavity, for growing a culture on the substrate;
wherein the cavity comprises an inlet for cell culture medium;
wherein the method comprises:
   supplying cell culture medium to the cavity through the inlet for growing the culture.

Thus, according to the second aspect, cultures, e.g. cell cultures or tissue structures, may be grown in the bioreactor chamber and supplied with a cell culture medium (e.g. including nutrients for cell growth). In some embodiments, the method comprises supplying a cell suspension to the substrate through the inlet for seeding cells (e.g. tissue cells) onto the substrate. In some embodiments, the substrate comprises a scaffold. Preferably the substrate (e.g. scaffold) is connected or fixed to the first end block and/or the second end block.

In one set of embodiments, the first end block and/or the second end block comprise an inlet for cell culture medium that is connected to a point proximal to the point at which the substrate (e.g. scaffold) is connected to or fixed to the first end block and/or the second end block. This helps to provide an effective supply of cell culture medium to the substrate (e.g. scaffold), and, if the inlet is used to supply cell suspension, this helps to allows a cell suspension to be supplied easily to the scaffold without the need for an additional inlet. However, in preferred embodiments, the method comprises seeding tissue cells into the substrate (e.g. scaffold) before construction of the bioreactor chamber. In at least preferred embodiments, the method further comprises maintaining conditions within the bioreactor chamber to be those suitable for cell or tissue culture (e.g. structure) growth.

In some embodiments, the cavity further comprises an outlet. In some embodiments, the method further comprises removing (e.g. used) cell culture medium from the cavity through the outlet. This helps to drain the cavity.

In some embodiments, the first end block and/or the second end block comprise a fixing point, suitable for connection to a mechanical actuator. The fixing point is preferably arranged on the first end block and/or the second end block so that, once the bioreactor chamber is assembled, the fixing point is located outside the cavity. In some embodiments, the fixing point comprises an attachment member, for example a ring, for connecting between the end block and a mechanical actuator. Optionally, only one of the first end block and the second end block are arranged to be moved by a mechanical actuator. This may allow the bioreactor chamber to be actuated by simply holding one of the end blocks in place, and actuating the other end block. The combination of a bioreactor chamber as described above, together with a mechanical actuator, provides a bioengineering system.

Thus, from a further aspect, the invention provides a bioengineering system, comprising:
a bioreactor chamber, comprising:
   a first end block;
   a second end block;
   a flexible membrane, extending between the first end block and the second end block, to define a cavity bounded by at least the flexible membrane;
   wherein the cavity is arranged to receive:
      a substrate, for growing a culture on the substrate; or
      a biomaterial for testing the biomaterial; and
   a mechanical actuator, connected to the first end block and/or the second end block;
   wherein the mechanical actuator is configured to actuate the first end block and/or the second end block.

While the system may comprise only a single mechanical actuator connected to the first and/or second end block, in one set of embodiments the system comprises a first mechanical actuator connected to, and configured to actuate, the first end block, and a second mechanical actuator connected to, and configured to actuate, the second end block. Preferably the mechanical actuator is a multi-directional actuator. Preferably the mechanical actuator is an actuator with six degrees of freedom. The six degrees of freedom refer to the actuator being capable of actuating translational movement along three perpendicular axes, and also being capable of actuating rotational movement about three perpendicular axes. Thus the mechanical actuator may be arranged to apply tension, compression, torsion and/or shear stresses to the cavity (and thus the substrate and the culture (e.g. cell or tissue culture (e.g. structure)) being grown thereon, or the biomaterial) via the first and/or second end blocks.

In some embodiments, the mechanical actuator is an actuator that is arranged to mimic human movement. Preferably, the mechanical actuator is arranged to mimic a specific joint (e.g. a shoulder or knee) in the human or animal body. This advantageously allows the bioreactor chamber to be connected to an actuator and, e.g., placed in a location on an actuatable structure (such as a humanoid robot), which closely mimics the conditions of a particular joint in the human or animal body.

Thus, if the bioreactor chamber is to be used to grow a particular tissue type, the bioreactor chamber may be connected to an actuator and, e.g., mounted on an actuatable structure, which most closely mimics the environment (specifically the mechanical stimulation) in the location in the human or animal body in which that tissue type would normally occur. The mechanical actuator may comprise (or form part of) a musculoskeletal humanoid robot. A musculoskeletal humanoid robot is a subclass of humanoid robots, which is specifically designed to mimic the musculoskeletal system of a human.

In some embodiments the bioengineering system may comprise a supply of cell culture medium, e.g. fluidly connected to the fluid inlet of the cavity, for supplying cell culture medium into the cavity.

The bioengineering system described above may be used for cell or tissue engineering. Thus, from a further aspect, the invention provides a method of growing a culture using a bioreactor chamber, wherein the bioreactor chamber comprises:
a first end block;
a second end block;
a flexible membrane, extending between the first end block and the second end block, to define a cavity bounded by at least the flexible membrane; and
a substrate, arranged within the cavity for growing a culture on the substrate;
wherein the cavity comprises an inlet for cell culture medium;
wherein the method comprises:
   supplying cell culture medium to the cavity of the bioreactor, through the inlet, for growing a culture;
   attaching the first end block and/or the second end block to a mechanical actuator; and
   moving the first end block and/or the second end block using the mechanical actuator, to move the substrate, so to apply mechanical stimulation to the culture being grown within the cavity.

In some embodiments, the first end block and/or the second end block, and any component parts, may be 3D printed or may be cast using a die or a mould. This helps to provide accurate part dimensions and shapes. The bioreactor chamber and its components may be any size that is suitable in size for the intended culture growth or biomaterial. In one example, the flexible member (in an untensioned state) defines a cavity which is less than 10 cm long, along its longest length, e.g. less than 5 cm long.

From a further aspect, the invention provides a biomaterial testing system for testing a biomaterial, comprising:
a first end block;
a second end block;
a flexible membrane, extending between the first end block and the second end block, to define a cavity bounded by at least the flexible membrane; and
a biomaterial arranged within the cavity.

Optionally, the biomaterial testing system further comprises a mechanical actuator, connected to the first end block and/or the second end block; wherein the mechanical actuator is configured to actuate the first end block and/or the second end block.

Features disclosed herein with reference to the earlier aspects may be optional features of embodiments of the other aspects also. Features of any aspect or embodiment described herein may, wherever appropriate, be applied to any other aspect or embodiment described herein. Where reference is made to different embodiments or sets of embodiments, it should be understood that these are not necessarily distinct but may overlap.

Certain preferred embodiments of the invention will now be described, by way of example only, with reference to the accompanying drawings, in which:

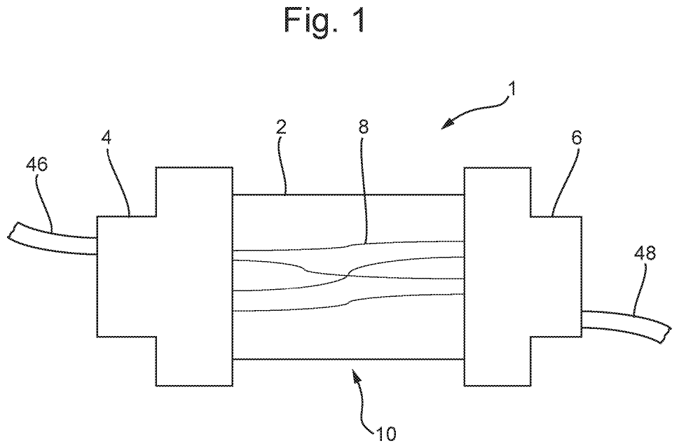

FIG. 1 is a schematic diagram showing the bioreactor chamber apparatus according to the an embodiment of present invention.

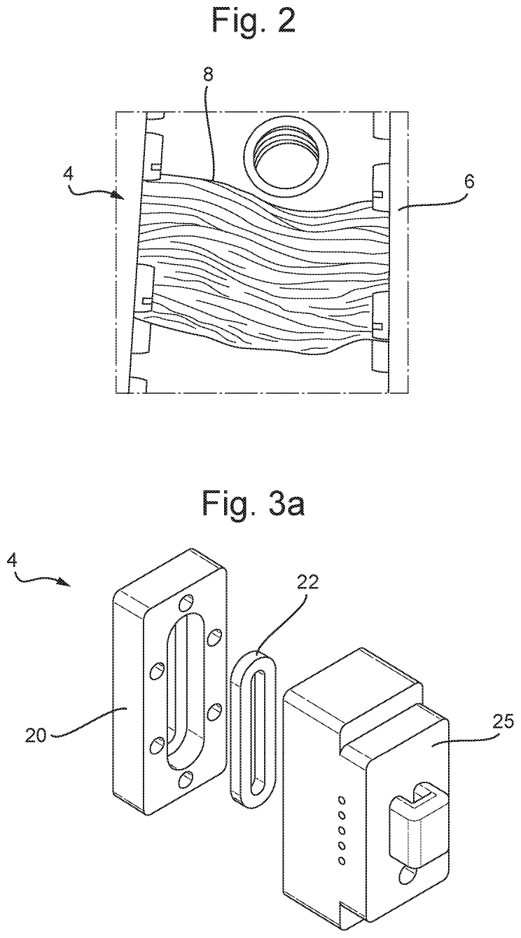

FIG. 2 is an image showing a scaffold, extending between a first end block and a second end block of a bioreactor chamber according to an embodiment of the present invention.

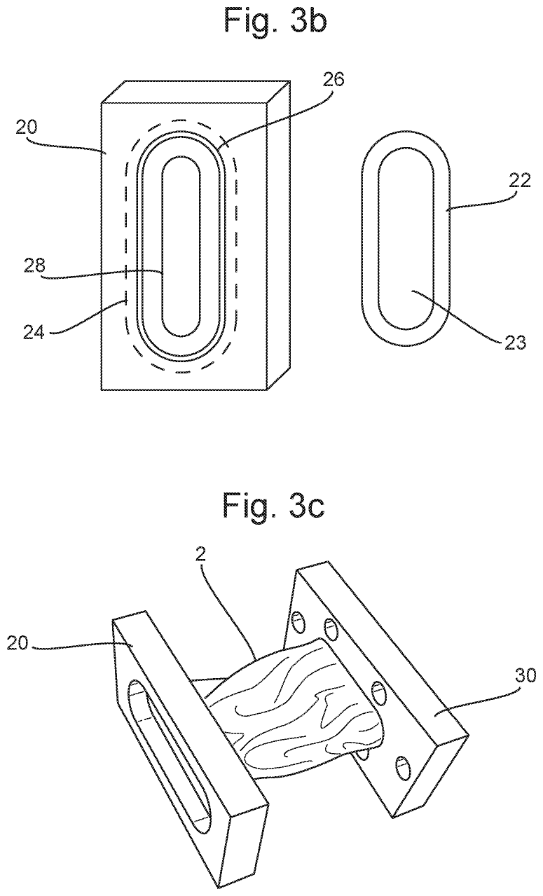

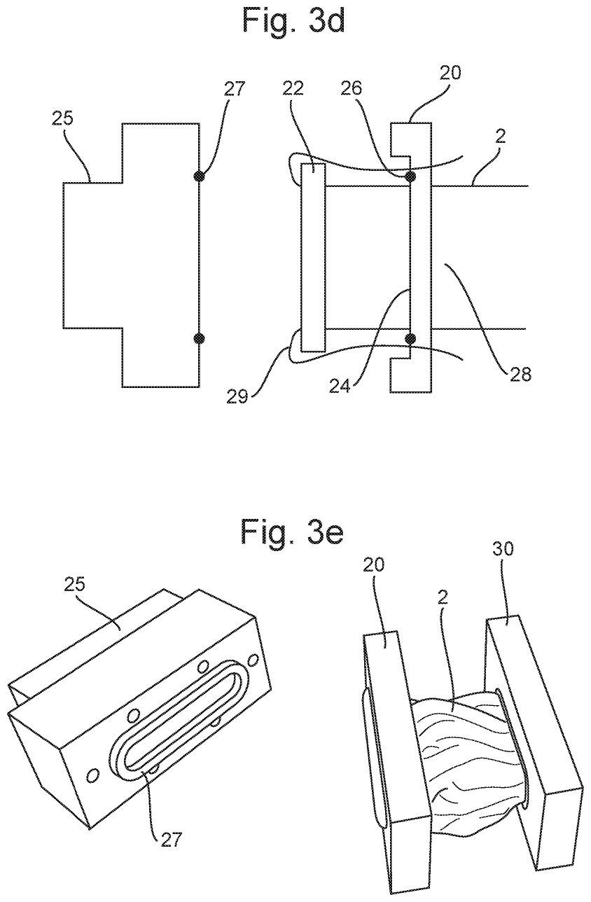

FIG. 3a is a three-dimensional exploded view of an outer member of an end block of a bioreactor chamber according to an embodiment of the present invention.

FIG. 3b is a schematic diagram showing an inner member and a rigid ring, which are parts of an end block of a bioreactor chamber according to an embodiment of the present invention.

FIG. 3c is an image showing two inner members, with a membrane extending between them, according to an embodiment of the present invention.

FIG. 3d is a cross-sectional view of a membrane connected within an end block of a bioreactor chamber, according to an embodiment of the present invention.

FIG. 3e is an image showing two inner members, with a membrane extending between them, together with an outer member, according to an embodiment of the present invention.

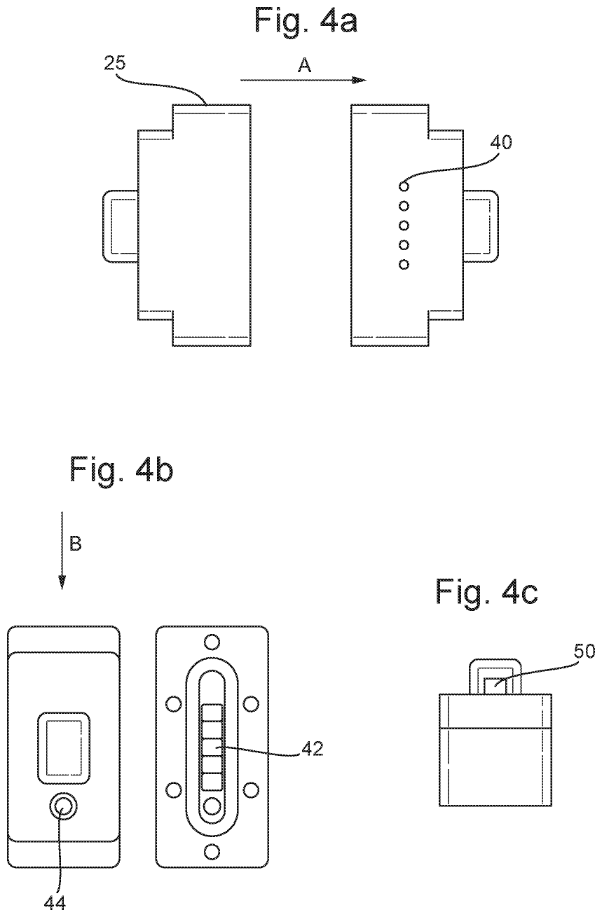

FIG. 4a is a schematic drawing showing a side view of an outer member of an end block of a bioreactor chamber according to an embodiment of the present invention.

FIG. 4b is a schematic drawing showing a view of an outer member of an end block of a bioreactor chamber according to an embodiment of the present invention, along the line A as shown in FIG. 4a.

FIG. 4c is a schematic drawing showing a view of an outer member of an end block of a bioreactor chamber according to an embodiment of the present invention, along the line B as shown in FIG. 4b.

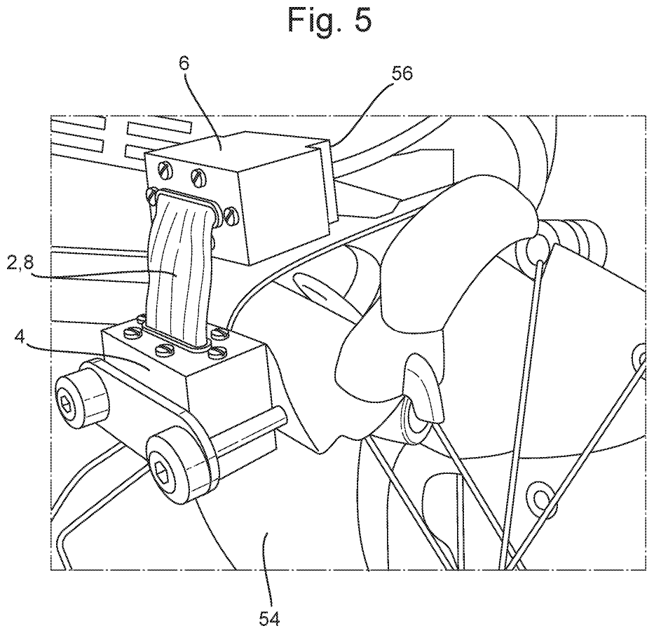

FIG. 5 is an image showing the bioreactor chamber according to an embodiment of the present invention, connected to a humanoid robot, specifically a humanoid robotic shoulder.

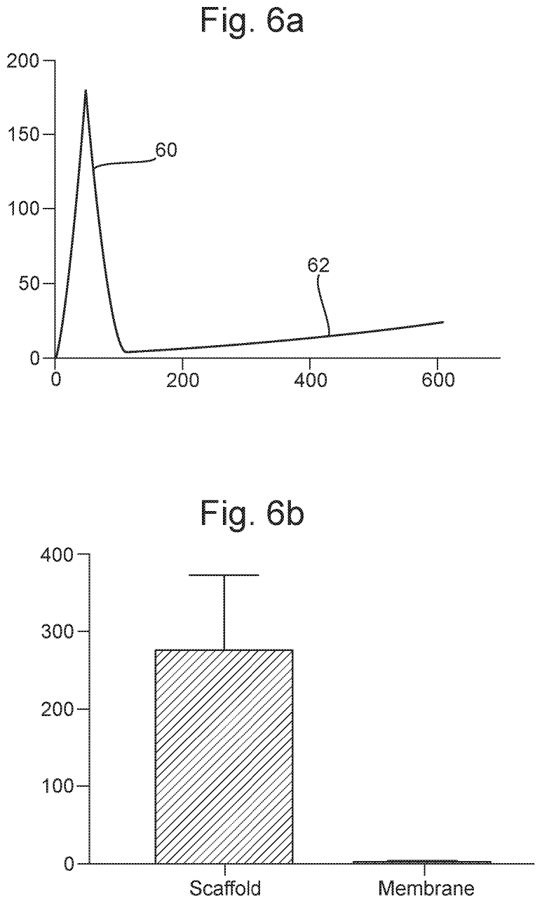

FIG. 6a is a graph showing the percentage strain (x-axis) against the stress in MPa (y-axis) as tensile stress is applied to a bioreactor chamber according to an embodiment of the present invention.

FIG. 6b is a graph showing the Young's modulus of an exemplary scaffold and flexible membrane according to an embodiment of the present invention, in units of MPa.

Throughout the figures, like reference numerals have been used for like elements.

Bioreactors, or bioreactor chambers, are used in tissue engineering to provide a controlled environment, in which conditions can be controlled to meet the requirements needed for maintaining and stimulating living cells and tissues outside the body. Robotic devices may be integrated into bioreactor chambers to provide mechanical cues to a growing tissue sample which modulate the growth of the tissue. Mechanical stresses (tension, compression, torsion and shear stresses) naturally occur in vivo and are crucial to the development and maintenance of musculoskeletal tissues such as tendons (soft tissues connecting muscles and bones).

Conventional bioreactor chambers are capable of applying only unilateral forces using a linear actuator which connects directly to a cell or tissue sample. This is unsatisfactory, since it is known that tendons undergo profound modifications when deprived of certain stresses, including a reduction of their anatomical size, a rapid deterioration of their mechanical properties and a change in extracellular matrix (ECM) composition and organisation. The existing evidence suggests that mimicking physiological stresses more closely would improve the quality of tendon grafts produced, however this is not currently possible due to the structure of available bioreactor chambers. Furthermore, in current bioreactor chamber designs the actuator is contained within the bioreactor chamber, and connected directly to the tissue sample, such that the sample cannot be separated from the actuation system without opening the bioreactor chamber and therefore risking contamination.

The aim of the present invention is therefore to provide an improved bioreactor chamber. A simple schematic drawing of the bioreactor chamber 1 according to an embodiment of the present invention is shown in FIG. 1.

The bioreactor chamber 1 comprises a flexible membrane 2 extending between a first end block 4 and a second end block 6. The flexible membrane 2, together with the first end block 4 and the second end block 6 defines a cavity 10. In the example of the Figures the substrate within the cavity is a scaffold 8. The scaffold 8 extends between the first end block 4 and the second end block 6, within the cavity 10. The membrane 2 surrounds the scaffold 8 and acts as the chamber's wall (i.e. the wall of the cavity 10) to contain the scaffold 8. In use, the cavity 10 may also contain a tissue engineering medium, supported by the scaffold 8, for growing a tissue sample, the cavity created by the flexible membrane 2, the first end block 4 and the second end block 6 is therefore important to maintain sterility inside the cavity 10 for the growth of a tissue sample. The membrane 2 comprises a thin sheet of transparent polyurethane rolled into a tubular shape and sealed along the long edge.

The embodiment of the present invention thus provides a bioreactor chamber 1 which is both flexible, owing to the chamber cavity 10 being formed, at least in part, by a flexible membrane 2, and which is independent from a surrounding mechanical actuation system (since the end blocks 4, 6 can be attached to a mechanical actuator, without any part of the actuator attaching directly to the scaffold 8). This improvement enables the application of multiaxial motions by complex actuation system, since torsion, compressive and tensile forces can easily be transmitted through the flexible membrane 2. The flexible membrane 2 also allows the tissue within the bioreactor chamber 1 to be easily observed (e.g. by transporting the entire sealed bioreactor chamber to a microscope) without interfering with the inner sample.

The scaffold 8 or artificial matrix is made of parallel PCL filaments produced by electrospinning, shown in more detail in FIG. 2. In this example, a total of 200 filaments, regrouped in 5 bundles of 40 filaments each, have been stretched through the tubular membrane and fixed in the end blocks at both ends with medical grade resin. Each filament is made of aligned submicron fibres having an average diameter of 800 nm. This arrangement creates a highly anisotropic and porous scaffold.

In the example shown in the Figures, the flexible membrane 2 is sealingly connected to each of the first end block 4, and the second end block 6. It is of course possible that at another point on the boundary of the cavity 10 (i.e. in one of the end blocks) an opening may be formed forming a connection from the exterior of the cavity to the interior of the cavity.

One example of a sealing connection between the membrane 2 and each end block 4, 6 is shown in FIGS. 3a-3e. The structure of an end block will now be described with reference to the first end block 4, however it will be understood by the skilled person that any of these features may, either additionally or alternatively, be present in the second end block 6. The first end block 4 comprises an inner member 20, a rigid ring 22 and an outer member 25, as shown in the three-dimensional exploded view of FIG. 3a.

The inner member 20 and the rigid ring 22 are shown in more detail in FIG. 3b. The inner member 20 comprises a recessed portion 24, indicated in FIG. 3b by a dashed line. The recessed portion 24 is of a suitable shape and size to accommodate the rigid ring 22. In order to provide a better seal, a first rubber O-ring 26 is provided within the recessed portion 24 of the inner member 20.

The flexible member 2 is shown in FIG. 3c, connected between the inner member 20 of the first end block 4 and the inner member 30 of the second end block 6. In order to form the sealing connection between the flexible member 2 and the first end block 4, i.e. to seal the flexible membrane 2 against the inner member 20, the flexible membrane 2 is passed through an aperture 28 in the inner member 20, from the side which does not contain the recessed portion 24, through the aperture 28, which opens, on the other side, into the recessed portion 24 of the inner member 20. The flexible membrane 2 is then passed through the central aperture 23 of the rigid ring 22, which is defined by the inner edge of the rigid ring 22. The flexible membrane 2, which in this example has a tubular shape i.e. a substantially circular or oval cross-section, is then folded back on itself, so as to surround the outer edge of the rigid ring 22.

The flexible membrane 2, which is surrounding the outer edge of the rigid ring 22, is then passed back through the aperture 28 in the inner member 20. The rigid ring 22 is then inserted into the recessed portion 24, which is sized to contain it, and is pushed against the first rubber O-ring 26 to form a seal. The resulting structure is shown in FIG. 3c, and is also shown in cross-section in FIG. 3d.

The rigid ring 22 may be fixedly attached to the inner member 20, for example it may be glued in place. Alternatively, as shown, the rigid ring 22 may be clamped in position, i.e. held in the sealing position by pressure, by fixing the outer member 25 to the inner member 22, retaining the rigid ring 22 between the inner member 20 and the outer member 25. As shown in FIG. 3d, a second rubber O-ring 27 may be provided between the outer member 25 and the rigid ring 22. The second rubber O-ring is positioned so that, when the outer member 25 and the inner member 20 are bought together, with the rigid ring 22 clamped between them, the second rubber O-ring 27 contacts the flexible membrane 2, at the point 29 at which the flexible membrane 2 passes around the exterior of the rigid ring 22. This advantageously provides an improved seal and reduces wear on the flexible membrane.

The outer member 25 may optionally be attached to the inner member 20 by inserting screws through screw holes in the inner member 20, and screwing these into the outer member 25. This clamps the rigid ring 22 against the two parts of the flexible membrane 2, which are passing through the aperture 28 in the inner member 20, in order to form a seal. This stage is also illustrated in FIG. 3e, which is an image showing both of the inner members 20, 30, with the membrane 2 connected between them.

The scaffold 8 may be placed inside the flexible membrane 2 before, or after, the flexible membrane 2 is attached to the rigid ring 22 and inner member 20. Preferably, the scaffold is placed within the cavity before the outer members of both of the end blocks are attached to the inner members of the respective end blocks, so as to clamp the membrane in place. FIG. 3e shows the outer member 25, including second rubber O-ring 27, ready to be connected to the first inner member 20, in order to clamp the flexible membrane 2 in place. This arrangement largely prevents slippage of the flexible membrane and also creates a seal so as to prevent leakage out of the cavity 10 at the point where the membrane meets the end blocks. Other points on the surface of the cavity may be arranged to provide inlets or outlets to the interior of the cavity 10.

In particular, in the example shown in FIG. 4a, the outer member 25 includes resin channels 40. The outer member 25 is shown in a side profile in FIG. 4a, the side being the profile which is also shown in FIG. 1. As shown the resin channels are only visible from one of the two sides of the outer member 25. The resin channels 40 connect from the side of the outer member 25, to the inner face of the outer member 25, i.e. the surface which is adjacent to the inner member 20 when the bioreactor chamber 1 is assembled. The resin channels 40 therefore form an inlet to the cavity 10, allowing resin to be added to the cavity.

FIG. 4b shows an “end-on” view of the outer member 25, i.e. a view along the line A shown in FIG. 4a, from both an outer side (left) and an inner side (right), the inner side being the side that, when the bioreactor chamber is assembled, is adjacent to the inner member 20. The surface of the outer member 25 shown on the right of FIG. 4b is the surface to which the resin channels 40 connect. As shown in FIG. 4b, the outer member 25 includes scaffold channels 42, into which the scaffold may be inserted. The scaffold channels 42 may be blind channels, meaning that they are closed at one end and do not form a connection between the cavity 10 and the external environment. In contrast, the resin channels 40 may be “go-through” channels which connect from the external environment to the cavity 10, allowing resin to be inserted into the cavity 10. Optionally the scaffold may be fixed within the scaffold channels 42. The resin channels 40 may connect to, or feed into, the scaffold channels 42, for example the resin channels 40 may run perpendicularly to the scaffold channels 42, and may meet the scaffold channels 42 perpendicularly, allowing resin to be injected into the end of the scaffold channels 42.

The outer member 25 additionally includes a tubing channel 44. An inlet tube 46 may be connected to the tubing channel of the outer member 25 of the first end block 4, as shown in FIG. 1. An outlet tube 48 may be connected to the tubing channel of the outer member of the second end block 6. The inlet and outlet tubes 46, 48 allow perfusion of the medium during culture. In a particularly advantageous arrangement, as shown in FIG. 1, the inlet tube 46 and the outlet tube 48 are arranged to be positioned offset from each other i.e. to be not directly opposite each other on opposing sides of the cavity. This forces the perfusion medium to flow across the scaffold 8, as it passes from the inlet tube 46 to the outlet tube 48.

FIG. 4b also shows that there is an attachment member 50 arranged on the exterior surface of the outer member 25. This is more clearly visible in FIG. 4c, which shows a view along the line B, as shown in FIG. 4b. The attachment member 50 may, for example, comprise a loop, formed on the exterior surface of the outer member 25, through which a clip, or string, or other suitable connection may be passed, so as to create a mechanical connection between the outer member 25 (and therefore the bioreactor chamber 1) and an actuator.

FIG. 5 is an image, showing the bioreactor chamber 1 of an embodiment of the present invention attached to a multi-directional actuator 52. In this example, the multi-directional actuator 52 is a real-size musculoskeletal (MSK) humanoid robot. The bioreactor chamber 1 is attached to a robotic arm of the humanoid robot. MSK humanoid robots are a class of humanoids that aim to replicate the human MSK system by mimicking the inner structures of the human body such as muscles, tendons and bones. Their actuators mimic the physiologic behaviour of muscles by pulling the skeletal structure using a series of strings. In this example, the first end block 4 is attached to the humerus 54 of the humanoid robotic arm, and the second end block 6 is attached to a muscle string 56 of the humanoid robotic arm, at a location which corresponds to that of the supraspinatus tendon.

It is particularly advantageous to certain aspects of the present invention that the scaffold 8 contributes to most of the load bearing effect of the bioreactor chamber 1 at low strains, compared to the flexible membrane 2 which contributes little. This is shown in the graph of FIG. 6a. FIG. 6a represents the percentage strain (x-axis) against the stress in MPa (y-axis) as tensile stress is applied to the bioreactor chamber. The scaffold contribution 60 can be seen in the sharp peak on the left of the graph, showing that the maximum failure stress of the scaffold was at approximately 170 MPa, with a corresponding maximum strain of around 75%. The contribution of the membrane 62 can be seen in the much gentler slope, appearing on the graph only at higher strains. The flexible membrane 2 maintains its integrity until approximately 500% of strain.

The graph of FIG. 6b shows the Young's modulus of the scaffold (left) and the membrane (right) in units of MPa (y-axis). This graph highlights the large difference between the Young's modulus of the scaffold 8 and the flexible membrane 2. This ensures that any mechanical actuation which the scaffold 8 is able to withstand can be tolerated by the flexible membrane 2 without any risk of damage or yielding.

It will be appreciated by those skilled in the art that the invention has been illustrated by describing one or more specific embodiments thereof, but is not limited to these embodiments; many variations and modifications are possible, within the scope of the accompanying claims. For example, while the Figures refer primarily to growing a cell culture (e.g. a tissue structure) in the cavity, it will be appreciated that the cavity may alternatively be used for testing a biomaterial, e.g. by placing the biomaterial in the cavity (e.g. connected between the first and second end blocks).

1. A bioreactor chamber, comprising:
a first end block;
a second end block; and
a flexible membrane, extending between the first end block and the second end block, to define a cavity bounded by at least the flexible membrane; and
wherein the cavity is arranged to receive:
a substrate, for growing a culture on the substrate; or
a biomaterial for testing the biomaterial.

2. The bioreactor chamber of claim 1, wherein the cavity is bounded by the first end block, the second end block and the flexible membrane.

3. The bioreactor chamber of claim 1, wherein the cavity has a substantially tubular shape.

4. The bioreactor chamber of claim 1, wherein the flexible membrane is at least partly transparent.

5. The bioreactor chamber of claim 1, wherein the flexible membrane is less than 100 microns thick.

6. The bioreactor chamber of claim 1, wherein the flexible membrane is sealingly connected to the first end block and/or the second end block.

7. The bioreactor chamber of claim 1, wherein the first end block and/or the second end block comprises an outer member and an inner member, and wherein the flexible member is clamped between the outer member and the inner member.

8. The bioreactor chamber of claim 7, wherein the first end block and/or the second end block comprises a ring; wherein the flexible membrane passes through an aperture in the inner member, through the ring, to be folded back on itself to surround the ring, and passes back through the aperture in the inner member; and wherein the flexible membrane, the outer member and the inner member are attached together, and the ring is clamped between the outer member and the inner member.

9. The bioreactor chamber of claim 8, wherein the inner member comprises a recessed portion, wherein the recessed portion further comprises the aperture, and wherein the recessed portion at least partially contains the ring.

10. (canceled)

11. The bioreactor chamber of claim 1, wherein the substrate comprises a scaffold and wherein the scaffold extends between the first end block and the second end block.

12. (canceled)

13. The bioreactor chamber of claim 1, wherein the substrate comprises a scaffold and wherein the scaffold comprises a plurality of substantially aligned electrospun filaments.

14. The bioreactor chamber of claim 1, wherein the first end block and/or the second end block comprise a fixing point, for connection to a mechanical actuator.

15. The bioreactor chamber of claim 1, wherein the cavity comprises an inlet for cell culture medium.

16. (canceled)

17. The bioreactor chamber of claim 15, wherein the first end block comprises the inlet and the second end block comprises an outlet, and wherein the inlet and the outlet are arranged to be positioned offset from each other on opposing sides of the cavity.

18. (canceled)

19. A bioengineering system, comprising:
a bioreactor chamber as claimed in claim 1;
a mechanical actuator, connected to the first end block and/or the second end block;
wherein the mechanical actuator is configured to actuate the first end block and/or the second end block.

20. The bioengineering system of claim 19, wherein the mechanical actuator is a multi-directional actuator.

21. The bioengineering system of claim 19, wherein the mechanical actuator is constructed to mimic a specific joint in the human or animal body.

22. A method of growing a culture comprises:
supplying cell culture medium to the cavity of a bioreactor chamber as claimed in claim 15, through the inlet, for growing a culture, wherein the bioreactor chamber comprises a substrate, arranged within the cavity, for growing the culture on the substrate;
attaching the first end block and/or the second end block to a mechanical actuator; and
moving the first end block and/or the second end block using the mechanical actuator, to move the substrate, so to apply mechanical stimulation to the culture being grown within the cavity.

23. The method of claim 22, wherein the mechanical actuator is a multi-directional actuator.

24. The method of claim 22, wherein the mechanical actuator is constructed to mimic a specific joint in the human or animal body.

25. (canceled)
